(19) United States
(12) Patent Application Publication
(10) Pub. No.: US 2021/0274924 A1
(43) Pub. Date: Sep. 9, 2021

(54) ELECTRIC TOILET BRUSH

(71) Applicant: Jinghan Zhang, Shenzhen (CN)

(72) Inventor: Jinghan Zhang, Shenzhen (CN)

(21) Appl. No.: 16/861,151

(22) Filed: Apr. 28, 2020

(30) Foreign Application Priority Data
Mar. 9, 2020 (CN) 202010156157.9

(51) Int. Cl. A46B 13/02; A46B 5/00; A46B 13/00; A46B 5/02; A47K 17/00

(52) U.S. Cl. CPC A46B 13/02; A46B 5/0095; A46B 2200/304; A46B 5/02; A47K 17/00; A46B 13/001

(57) ABSTRACT

The invention discloses an electric toilet brush comprising handle, elongate rod, deformed connecting section, handpiece, and brush head connected orderly; said handpiece includes mounted housing, motor bracket, motor, planetary reduction gearbox and bearing; mounted housing is a horn-shaped hollow shell with smaller opening connected with deformed connecting section and bigger opening mounted with motor bracket whose side facing mounted housing is provided with motor while other side provided with planetary reduction gearbox connected with motor; dismountable brush head can join to outside of bearing and wrap planetary reduction gearbox in brush head; the deformation piece, arranged between elongate rod and handpiece, can deform and clean; cooperation between dismountable brush head and bearing enables brush head to wrap whole reduction gearbox; after pressing power button, the motor drives reduction gearbox to rotate which drives dismountable brush head to rotate; uv gemicidal light can sterilize by direct radiation on brush head.

### BACKGROUND OF THE INVENTION

### 1. Field of the Invention

The invention relates to the technical field of daily use articles, specifically relating to an electric toilet brush.

### 2. Description of the Related Art

The toilet brush is a cleaning device generally used for cleaning toilet or bathroom. Spotlessly clean in the toilet can effectively curb growth of bacteria, fungi, mould; However, at present, most of toilet brushes on the market consist of a straight rod and a brush head, which is not convenient to use and hard to satisfy usage requirement, tend to cause inconvenience in the using process. Accordingly, it has been an urgent problem that needs solving for those skilled in the art to design a convenient and user-friendly electric toilet brush.

### SUMMARY OF THE INVENTION

To solve above technical problems, the invention adopts following technical plan: an electric toilet brush comprising a handle, an elongate rod, a deformed connecting section, a handpiece, and a brush head connected in sequence; said handpiece includes a mounted housing, a motor bracket, a motor, a planetary reduction gearbox and a bearing wherein the mounted housing is a horn-shaped hollow shell whose smaller open end is connected with the deformed connecting section and whose bigger open end is mounted with the motor bracket; the side of said motor bracket that faces the mounted housing is provided with the motor while the other side is provided with the planetary reduction gearbox connected with the motor; one end of the motor bracket connected with the mounted housing is provided with a waterproof ring while the other end thereof is positioned at the outer side of the handpiece and is connected with the bearing; said brush head can be dismounted and joined to the outside of the bearing and wrap up the planetary reduction gearbox in the brush head;

as an improvement, the input end of said planetary reduction gearbox is connected with the brush head connector which is inserted into the brush head and connected with it.

As an improvement, said planetary reduction gearbox includes a reduction gearbox housing and a second stage planetary gear, a second stage planet carrier, a planet carrier fixing piece, a first stage planetary gear, a first stage planet carrier and an output shaft provided in the reduction gearbox housing, among which said output shaft extends out of the reduction gearbox housing and is connected with the brush head connector.

As an improvement, the second stage planetary gear passes through the second stage planet carrier and the planet carrier fixing piece to be engaged with the first stage planetary gear, which is drive-connected to the output shaft via first stage planetary gear; both of said first stage planetary gear and second stage planetary gear are three inter-engaged gears and are respectively installed with the first stage planet carrier and second stage planet carrier cooperatively via gear-mounting shafts; the lower part of the second stage planet carrier is provided with a gear shaft which is engaging transmitted with the first stage planetary gear and the lower part of the planet carrier fixing piece is provided with a gear mount that cooperates with the first stage planetary gear.

As an improvement, said motor bracket comprises a bracket ring sheathed out of the planetary reduction gearbox and a bracket plate arranged at an opening of the larger end of the mounted housing among which said bracket ring is arranged in the middle of the bracket plate in a penetrating mode; said bearing is arranged on the outer wall of the bracket ring and a plurality of uv germicidal lamps capable of irradiating the brush head are arranged on the outer edge of the bracket plate.

As an improvement, said deformed connecting section comprises a connecting elongate rod, a deformation piece of the handpiece, and a soft rubber sleeve arranged out of the deformation piece.

As an improvement, two ends of said soft rubber sleeve are provided with elongate sleeves which are respectively wrapped in the ends of the elongate rod and the handpiece.

As an improvement, said elongate rod is a telescopic rod or a fixing straight rod and at the outside of said handle is provided with a power button within which is set with a lithium battery; said lithium battery, the motor, the uv germicidal lamps and the power button form a circuit.

By adopting above structure, the invention has following merits: the apparatus is equipped with the deformation piece between elongate rod and the handpiece which can deform and clean in 360 degrees without dead angle under the effect of deformation piece; the cooperation between the dismountable brush head and bearing enables brush head to wrap whole reduction gearbox; after pressing the power button, the motor drives the reduction gearbox to rotate and the reduction gearbox drives the dismountable brush head to rotate; the invention has uv gemicidal light which can sterilize by direct radiation on the brush head after opening the power. The invention is reasonable in structure, convenient in usage, and suitable for popularization and application.

### BRIEF DESCRIPTION OF THE DRAWINGS

FIG. 1 is a diagram illustrating the breakdown structure of the electric toilet brush in the invention.

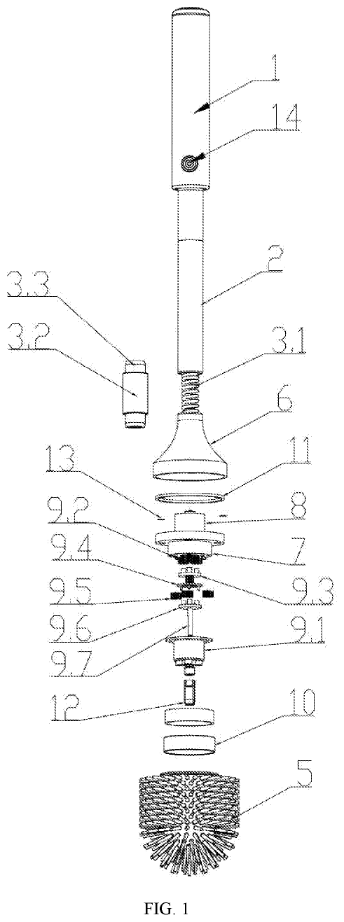

FIG. 2 is a diagram illustrating the connection structure of the electric toilet brush in the invention.

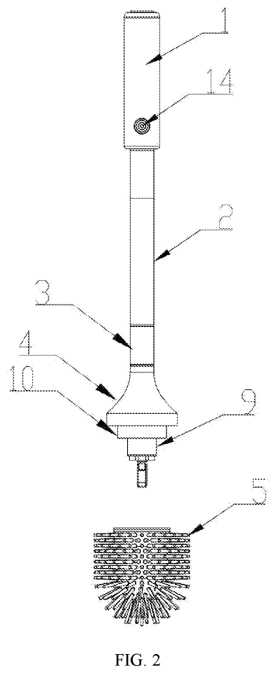

FIG. 3 is a diagram illustrating the external structure of the electric toilet brush in the invention.

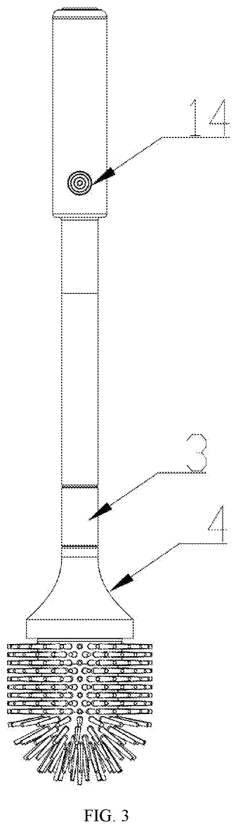

FIG. 4 is a structural diagram illustrating when the brush head of the electric toilet brush is deformed in the invention.

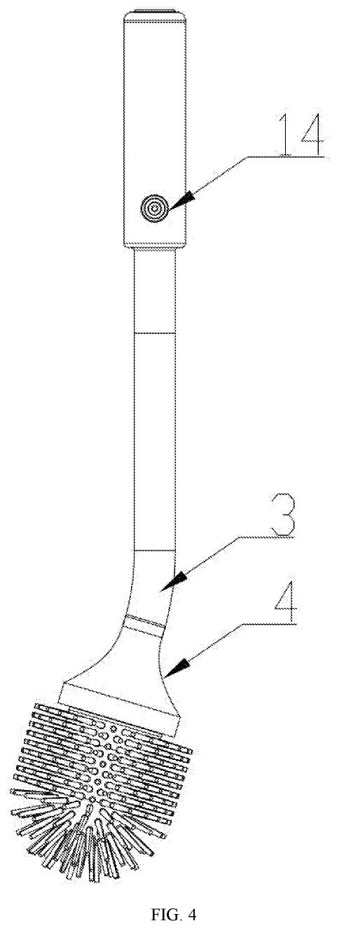

FIG. 5 is a a structural diagram illustrating the motor bracket of the electric toilet brush in the invention.

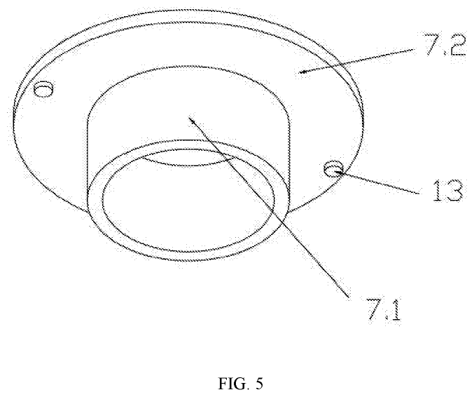

FIG. 6 is a structural diagram illustrating the planetary reduction gearbox of the electric toilet brush in the invention.

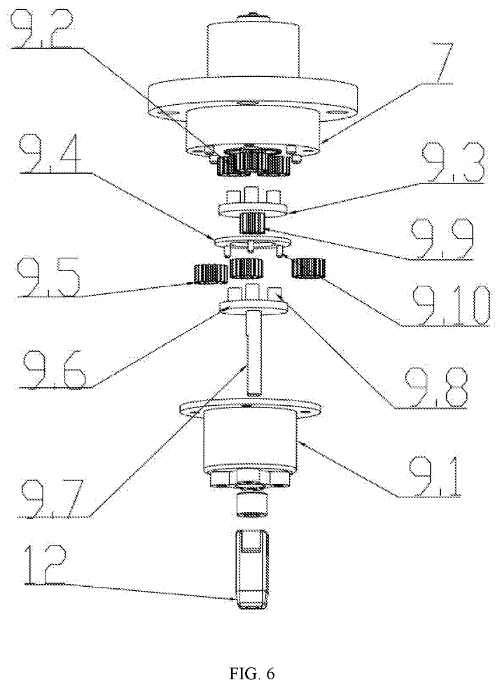

As shown in the drawing, 1. handle, 2. elongate rod, 3. deformed connecting section, 3.1. deformation piece, 3.2. soft rubber sleeve, 3.3. elongate sleeve, 4. handpiece, 5. brush head, 6. mounting housing, 7. motor bracket, 7.1. bracket ring, 7.2. bracket plate, 8. motor, 9. planetary reduction gearbox, 9.1. reduction gearbox housing, 9.2. second stage planetary gear, 9.3. second stage planet carrier, 9.4. planet carrier fixing piece, 9.5. first stage planetary gear, 9.6. first stage planet carrier, 9.7. output shaft, 9.8. gear-mounting shaft, 9.9. gear shaft, 9.10. gear mount, 10. bearing, 11. waterproof ring, 12. brush head connector, 13. uv gemicidal lamp, 14. power button.

### DETAILED DESCRIPTION OF THE PREFERRED EMBODIMENTS

With reference to the drawings, an electric toilet brush comprising a handle 1, an elongate rod 2, a deformed connecting section 3, a handpiece 4, and a brush head 5 connected in sequence; said handpiece 4 includes a mounted housing 6, a motor bracket 7, a motor 8, a planetary reduction gearbox 9 and a bearing 10 wherein the mounted housing 6 is a horn-shaped hollow shell whose smaller open end is connected with the deformed connecting section 3 and whose bigger open end is mounted with the motor bracket 7; the side of said motor bracket 7 that faces the mounted housing 6 is provided with the motor 8 while the other side is provided with the planetary reduction gearbox 9 connected with the motor 8; one end of the motor bracket 7 connected with the mounted housing 6 is provided with a waterproof ring 11 while the other end thereof is positioned at the outer side of the handpiece 5 and is connected with the bearing 10; said brush head 5 can be dismounted and joined to the outside of the bearing 10 and wrap up the planetary reduction gearbox 9 in the brush head 5.

As a preferred solution of the embodiment, the input end of said planetary reduction gearbox 9 is connected with the head connector 12 which is inserted into the brush head 5 and connected with it.

As a preferred solution of the embodiment, said planetary reduction gearbox 9 includes a reduction gearbox housing 9.1 and a second stage planetary gear 9.2, a second stage planet carrier 9.3, a planet carrier fixing piece 9.4, a first stage planetary gear 9.5, a first stage planet carrier 9.6 and an output shaft 9.7 provided in the reduction gearbox housing 9.1, among which said output shaft 9.7 extends out of the reduction gearbox housing 9.1 and is connected with the brush head connector 12.

As a preferred solution of the embodiment, the second stage planetary gear 9.2 passes through the second stage planet carrier 9.3 and the planet carrier fixing piece 9.4 to be engaged with the first stage planetary gear 9.5, which is drive-connected to the output shaft 9.7 via first stage planetary gear 9.6; both of said first stage planetary gear 9.5 and second stage planetary gear 9.2 are three inter-engaged gears and are respectively installed with the first stage planet carrier 9.6 and second stage planet carrier 9.3 cooperatively via gear-mounting shafts 9.8; the lower part of the second stage planet carrier 9.3 is provided with a gear shaft 9.9 which is engaging transmitted with the first stage planetary gear 9.9 and the lower part of the planet carrier fixing piece 9.4 is provided with a gear mount 9.10 that cooperates with the first stage planetary gear 9.5.

As a preferred solution of the embodiment, said motor bracket 7 comprises a bracket ring 7.1 sheathed out of the planetary reduction gearbox 9 and a bracket plate 7.2 arranged at an opening of the larger end of the mounted housing 6 among which said bracket ring 7.1 is arranged in the middle of the bracket plate 7.2 in a penetrating mode; said bearing 10 is arranged on the outer wall of the bracket ring 7.1 and a plurality of uv germicidal lamps 13 capable of irradiating the brush head 5 are arranged on the outer edge of the bracket plate 7.2.

As a preferred solution of the embodiment, said deformed connecting section comprises a connecting elongate rod, a deformation piece of the handpiece, and a soft rubber sleeve arranged out of the deformation piece.

As a preferred solution of the embodiment, two ends of said soft rubber sleeve 3.2 are provided with elongate sleeves 3.3 which are respectively wrapped in the ends of the elongate rod 2 and the handpiece 4.

As a preferred solution of the embodiment, said elongate rod 2 is a telescopic rod or a fixing straight rod and at the outside of said handle is provided with a power button 14 within which is set with a lithium battery; said lithium battery, the motor 8, the uv germicidal lamps 13 and the power button 14 form a circuit.

In particular implementation of the invention: In the handle is provided with the lithium battery and control plate; the deformation piece is arranged between elongate rod and the handpiece which can deform and clean in 360 degrees without dead angle under the effect of deformation piece; the planetary reduction gearbox is mounted in the reduction gearbox housing, the bearing is mounted on the motor bracket and the brush head connector is connected with the output shaft; the cooperation between the dismountable brush head and bearing enables brush head to wrap whole reduction gearbox; after pressing the power button, the motor drives the reduction gearbox to rotate and the reduction gearbox drives the dismountable brush head to rotate; the invention has uv gemicidal light which can sterilize by direct radiation on the brush head after opening the power. The brush head is connected with the bearing by snap connection in a dismountale mode; the motor is connected with the front end of the brush head via brush head connector extending into the brush head and is connected with the rear end thereof via the bearing to realize balanced and stable run between the brush head and the motor.

The invention and its embodiments have been described above, but the description is not limited thereto; only one embodiment of the invention is shown in the drawings, and the actual structure is not limited thereto. In general, it is to be understood by those skilled in the art that non-creative design of structural forms and embodiments that are similar to the technical solutions without departing from the spirit of the invention shall all fall within the protective scope of the invention.

1. An electric toilet brush comprising: a handle, an elongate rod, a deformed connecting section, a handpiece, and a brush head connected in sequence; said handpiece includes a mounted housing, a motor bracket, a motor, a planetary reduction gearbox and a bearing wherein the mounted housing is a horn-shaped hollow shell whose smaller open end is connected with the deformed connecting section and whose bigger open end is mounted with the motor bracket; the side of said motor bracket that faces the mounted housing is provided with the motor while the other side is provided with the planetary reduction gearbox connected with the motor; one end of the motor bracket connected with the mounted housing is provided with a waterproof ring while the other end thereof is positioned at the outer side of the handpiece and is connected with the bearing; said brush head can be dismounted and joined to the outside of the bearing and wrap up the planetary reduction gearbox in the brush head;

2. The electric toilet brush in claim 1 wherein the input end of said planetary reduction gearbox is connected with the brush head connector which is inserted into the brush head and connected with it.

3. The electric toilet brush in claim 2 wherein said planetary reduction gearbox includes a reduction gearbox housing and a second stage planetary gear, a second stage planet carrier, a planet carrier fixing piece, a first stage planetary gear, a first stage planet carrier and an output shaft provided in the reduction gearbox housing, among which said output shaft extends out of the reduction gearbox housing and is connected with the brush head connector.

4. The electric toilet brush in claim 3 wherein the second stage planetary gear passes through the second stage planet carrier and the planet carrier fixing piece to be engaged with the first stage planetary gear, which is drive-connected to the output shaft via first stage planetary gear; both of said first stage planetary gear and second stage planetary gear are three inter-engaged gears and are respectively installed with the first stage planet carrier and second stage planet carrier cooperatively via gear-mounting shafts; the lower part of the second stage planet carrier is provided with a gear shaft which is engaging transmitted with the first stage planetary gear and the lower part of the planet carrier fixing piece is provided with a gear mount that cooperates with the first stage planetary gear.

5. The electric toilet brush in claim 1 wherein said motor bracket comprises a bracket ring sheathed out of the planetary reduction gearbox and a bracket plate arranged at an opening of the larger end of the mounted housing among which said bracket ring is arranged in the middle of the bracket plate in a penetrating mode; said bearing is arranged on the outer wall of the bracket ring and a plurality of uv germicidal lamps capable of irradiating the brush head are arranged on the outer edge of the bracket plate.

6. The electric toilet brush in claim 1 wherein said deformed connecting section comprises a connecting elongate rod, a deformation piece of the handpiece, and a soft rubber sleeve arranged out of the deformation piece.

7. The electric toilet brush in claim 6 wherein two ends of said soft rubber sleeve are provided with elongate sleeves which are respectively wrapped in the ends of the elongate rod and the handpiece.

8. The electric toilet brush in claim 1 wherein said elongate rod is a telescopic rod or a fixing straight rod and at the outside of said handle is provided with a power button within which is set with a lithium battery; said lithium battery, the motor, the uv germicidal lamps and the power button form a circuit.
